(19) United States
(12) Patent Application Publication
(10) Pub. No.: US 2023/0059910 A1
(43) Pub. Date: Feb. 23, 2023

(54) Selectively Scent Infusible Face Masking Assembly

(71) Applicant: Mark Heger, Moville, IA (US)

(72) Inventor: Mark Heger, Moville, IA (US)

(21) Appl. No.: 17/405,587

(22) Filed: Aug. 18, 2021

(51) Int. Cl. A62B 23/02; A41D 13/11; B01D 46/00; A62B 18/08; A61L 9/12

(52) U.S. Cl. CPC A62B 23/025; A41D 13/1107; B01D 46/0038; A62B 18/08; A62B 18/084; A61L 9/12; A61L 2209/14

(57) ABSTRACT

A selectively scent infusible face masking assembly for filtering and scenting air includes a facemask, a pocket panel, and a plurality of pads. The facemask is selectively positionable over a nose and mouth of a user so that the facemask is removably engaged to a head of the user. The facemask filters particulates from air passing through the facemask. The pocket panel has opposing edges and a bottom edge that are engaged to the facemask to define a pouch. Each pad is infused with a respective aroma compound so that the plurality of pads comprises pads that are infused with a variety of aroma compounds. The pads are sized complementarily to the pouch. A respective pad is selectively insertable into the pouch so that the respective pad releases an associated aroma compound into the air passing through the facemask.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to face masking assemblies and more particularly pertains to a new face masking assembly for filtering and scenting air. The present invention discloses a face masking assembly comprising an air filtering facemask with a pouch disposed on its outer surface that is used to receive an aroma compound impregnated pad to provide a scent to inhaled air.

### (2) Description of Related Art Including Information Disclosed Under 37 CFR 1.97 and 1.98

The prior art relates to face masking assemblies. Prior art face masking assemblies designed to provide scented are comprise facemasks to which are engaged breakable capsules, scented patches, scented strips, scented objects, scented pods, and the like, which are attached to an inner surface of the facemask. What is lacking in the prior art is a face masking assembly comprising an air filtering facemask with a pouch disposed on its outer surface that is used to receive an aroma compound impregnated pad to provide a scent to inhaled air.

### BRIEF SUMMARY OF THE INVENTION

An embodiment of the disclosure meets the needs presented above by generally comprising a facemask, a pocket panel, and a plurality of pads. The facemask is configured to be selectively positioned over a nose and mouth of a user so that the facemask is removably engaged to a head of the user. The facemask is configured to filter particulates from air passing through the facemask. The pocket panel has opposing edges and a bottom edge that are engaged to the facemask to define a pouch. Each pad is infused with a respective aroma compound so that the plurality of pads comprises pads that are infused with a variety of aroma compounds. The pads are sized complementarily to the pouch. A respective pad is selectively insertable into the pouch so that the respective pad is configured to release an associated aroma compound into the air passing through the facemask.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a selectively scent infusible face masking assembly according to an embodiment of the disclosure.

FIG. 2 is a front view of an embodiment of the disclosure.

FIG. 3 is a rear view of an embodiment of the disclosure.

FIG. 4 is a cross-sectional view of an embodiment of the disclosure.

FIG. 5 is an in-use view of an embodiment of the disclosure.

FIG. 6 is a flow diagram for a method utilizing an embodiment of the disclosure.

### DETAILED DESCRIPTION OF THE INVENTION

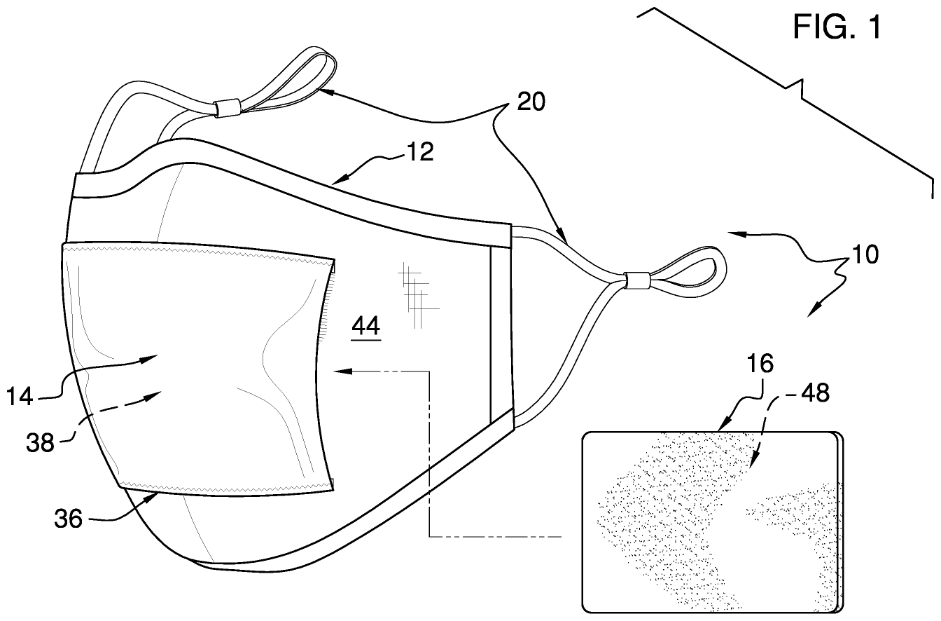

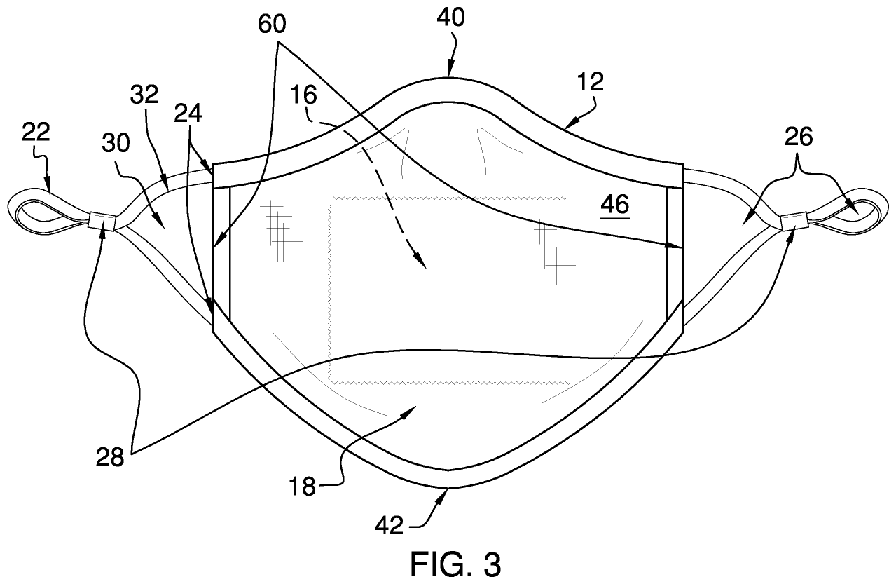

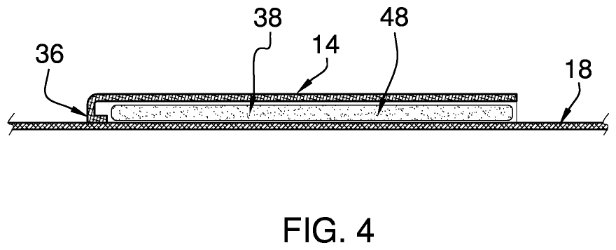

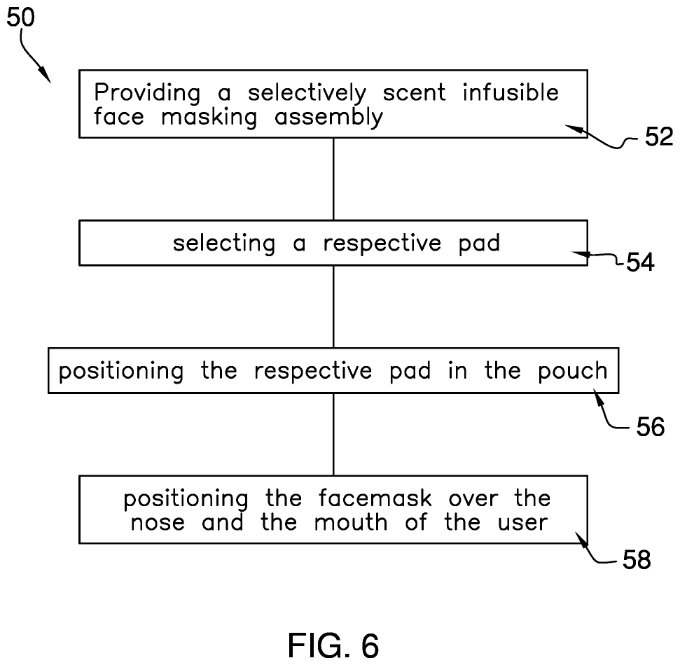

With reference now to the drawings, and in particular to FIGS. 1 through 6 thereof, a new face masking assembly embodying the principles and concepts of an embodiment of the disclosure and generally designated by the reference numeral 10 will be described.

As best illustrated in FIGS. 1 through 6, the selectively scent infusible face masking assembly 10 generally comprises a facemask 12, a pocket panel 14, and a plurality of pads 16. The facemask 12 is configured to be selectively positioned over a nose and mouth of a user so that the facemask 12 is removably engaged to a head of the user. The facemask 12 is configured to filter particulates from air passing through the facemask 12.

The facemask 12 comprises a face panel 18 and a pair of fasteners 20. The face panel 18 comprises one or more of polypropylene, cloth, paper, non-woven fabric, microfibers, and the like. Each fastener 20 is engaged to a respective opposed edge 60 of the face panel 18 and is configured to engage the head of the user so that the face panel 18 is removably engaged to the user.

The fastener 20 may comprise a strap 22, which has opposed ends 24 that are engaged to the respective opposed edge 20 to define a loop 26. The loop 26 is configured to be positioned around an associated ear of the user. Each of a pair of clips 28 may be slidably engaged to a respective strap 22 to define a subloop 30. The clip 28 is selectively and fixedly engageable to the respective strap 22 to fix a circumference 32 of the subloop 30. Fixing the circumference 32 of the subloop 30 effectively sizes the straps 22 to fit the facemask 12 to the user. The present invention anticipates other fastening means for engaging the facemask 12 to the head of the user, such as, but not limited to, elastic bands, clips, and the like.

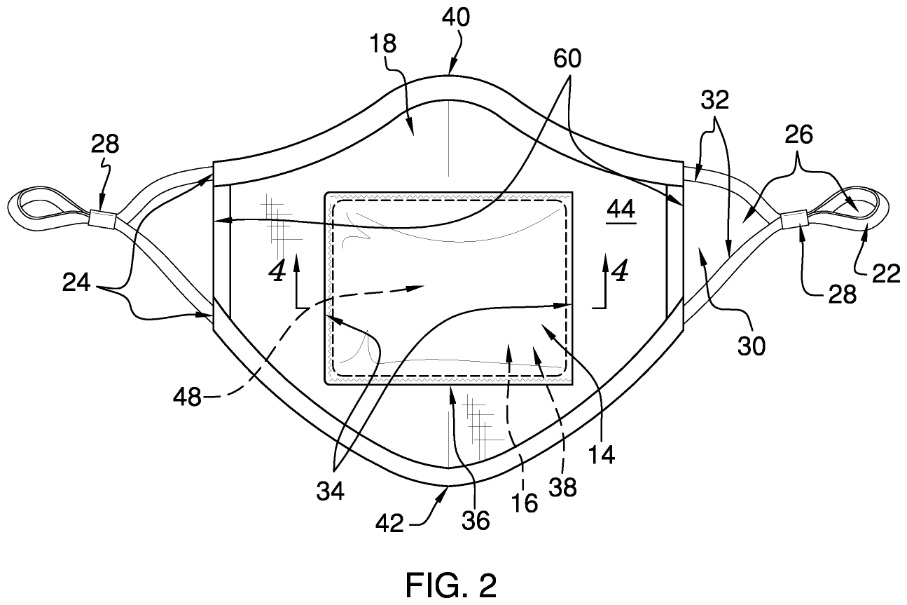

The pocket panel 14 has opposing edges 34 and a bottom edge 36 that are engaged to the facemask 12 to define a pouch 38. The pocket panel 14 may be substantially concentric with the facemask 12, as shown in FIG. 2, although the present invention anticipates the pocket panel 14 being biased to a respective opposed edge 50, an upper edge 40, or a lower edge 42 of the face panel 18. The pocket panel 14 may be engaged to an outer surface 44 of the facemask 12, as shown in FIG. 2, although the present invention anticipates the pocket panel 14 being engaged to an inner surface 46 of the facemask 12.

Each pad 16 is infused with a respective aroma compound 48 so that the plurality of pads 16 comprises pads 16 that are infused with a variety of aroma compounds 48. The pads 16 are sized complementarily to the pouch 38. A respective pad 16 is selectively insertable into the pouch 38 so that the respective pad 16 is configured to release an associated aroma compound 48 into the air passing through the facemask 12.

The present invention is anticipated to be useful in filtering particulates from air breathed by the user, such as dust, bacteria, and viruses. Additionally, the facemask 12 provides protection to the user and to persons proximate to the user by stopping aerosols and droplets, which can contain bacteria and viruses. Another anticipated benefit of the scent infusible face masking assembly 10 is provided by the aroma compound 48, which can mask a foul odor.

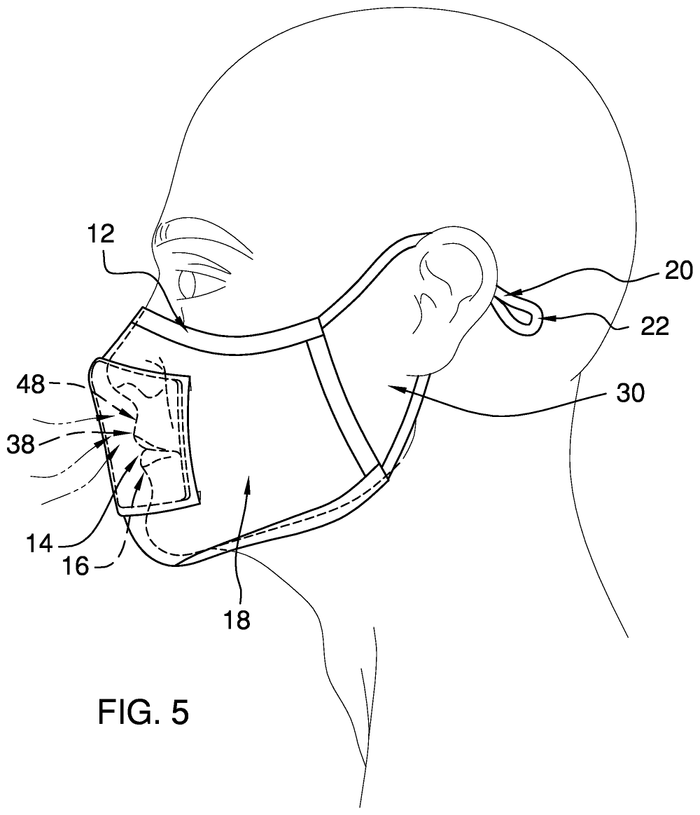

In use, the selectively scent infusible face masking assembly 10 enables a method 50 of filtering and scenting air. The method 50 comprises a first step 52 of providing a selectively scent infusible face masking assembly 10 according to the specification above. A second step 54 of the method 50 is selecting a respective pad 16. A third step 56 of the method 50 is positioning the respective pad 16 in the pouch 38, as shown in FIG. 1. A fourth step 58 of the method 50 is positioning the facemask 12 over the nose and the mouth of the user so that the facemask 12 is removably engaged to the head of the user, as shown in FIG. 5. The facemask 12 filters particulates from air passing therethrough while the aroma compound 48 is released into the air from the respective pad 16.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A selectively scent infusible face masking assembly comprising:
a facemask configured for selectively positioning over a nose and mouth of a user, such that the facemask is removably engaged to a head of the user, wherein the facemask is configured for filtering particulates from air passing through the facemask;
a pocket panel having opposing edges and a bottom edge engaged to the facemask defining a pouch; and
a plurality of pads, each pad being infused with a respective aroma compound, such that the plurality of pads comprises pads infused with a variety of aroma compounds, the pads being sized complementarily to the pouch, such that a respective pad is selectively insertable into the pouch, wherein the respective pad is configured for releasing an associated aroma compound into the air passing through the facemask.

2. The selectively scent infusible face masking assembly of claim 1, wherein the facemask comprises:
a face panel; and
a pair of fasteners, each fastener being engaged to a respective opposed edge of the face panel, the fastener being configured for engaging the head of the user, such that the face panel is removably engaged to the user.

3. The selectively scent infusible face masking assembly of claim 2, wherein the face panel comprises one or more of polypropylene, cloth, paper, non-woven fabric, and microfiber.

4. The selectively scent infusible face masking assembly of claim 2, wherein the fastener comprises a strap, the strap having opposed ends engaged to the respective opposed edge defining a loop, wherein the loop is configured for positioning around an associated ear of the user.

5. The selectively scent infusible face masking assembly of claim 4, further including a pair of clips, each clip being slidably engaged to a respective strap defining a subloop, the clip being selectively fixedly engageable to the respective strap for fixing a circumference of the subloop.

6. The selectively scent infusible face masking assembly of claim 1, wherein the pocket panel is substantially concentric with the facemask.

7. The selectively scent infusible face masking assembly of claim 1, wherein the pocket panel is engaged to an outer surface of the facemask.

8. A method filtering and scenting air, the method comprising the steps of:
providing a selectively scent infusible face masking assembly comprising:
a facemask configured for selectively positioning over a nose and mouth of a user, such that the facemask is removably engaged to a head of the user, wherein the facemask is configured for filtering particulates from air passing through the facemask,
a pocket panel having opposing edges and a bottom edge engaged to the facemask defining a pouch, and
a plurality of pads, each pad being infused with a respective aroma compound, such that the plurality of pads comprises pads infused with a variety of aroma compounds, the pads being sized complementarily to the pouch, such that a respective pad is selectively insertable into the pouch, wherein the respective pad is configured for releasing an associated aroma compound into the air passing through the facemask;
selecting a respective pad;
positioning the respective pad in the pouch; and
positioning the facemask over the nose and the mouth of the user, such that the facemask is removably engaged to the head of the user.

9. A selectively scent infusible face masking assembly comprising:
a facemask configured for selectively positioning over a nose and mouth of a user, such that the facemask is removably engaged to a head of the user, wherein the facemask is configured for filtering particulates from air passing through the facemask, the facemask comprising:
a face panel, the face panel comprising one or more of polypropylene, cloth, paper, non-woven fabric, and microfibers,
a pair of fasteners, each fastener being engaged to a respective opposed edge of the face panel, the fastener being configured for engaging the head of the user, such that the face panel is removably engaged to the user, the fastener comprising a strap, the strap having opposed ends engaged to the respective opposed edge defining a loop, wherein the loop is configured for positioning around an associated ear of the user, and
a pair of clips, each clip being slidably engaged to a respective strap defining a subloop, the clip being selectively fixedly engageable to the respective strap for fixing a circumference of the subloop;
a pocket panel having opposing edges and a bottom edge engaged to the facemask defining a pouch, the pocket panel being substantially concentric with the facemask, the pocket panel being engaged to an outer surface of the facemask; and
a plurality of pads, each pad being infused with a respective aroma compound, such that the plurality of pads comprises pads infused with a variety of aroma compounds, the pads being sized complementarily to the pouch, such that a respective pad is selectively insertable into the pouch, wherein the respective pad is configured for releasing an associated aroma compound into the air passing through the facemask.
